(19) United States
(12) Patent Application Publication
(10) Pub. No.: US 2025/0367096 A1
(43) Pub. Date: Dec. 4, 2025

(54) STABILIZATION OF THIOPYRIDINONE COMPOUND AND COMPOSITION COMPRISING SAME

(71) Applicant: L'OREAL, PARIS (FR)

(72) Inventors: Jun SUZUKI, Kawasaki-shi (JP); Makoto SAITO, Kawasaki-shi (JP); Yuan LUO, Kawasaki-shi (JP); Noemie BROMBERGER, Kawasaki-shi (JP); Romain TACHON, Kawasaki-shi (JP)

(21) Appl. No.: 18/874,784

(22) PCT Filed: Jun. 9, 2023
(86) PCT No.: PCT/JP2023/022336

§ 371 Date: Dec. 13, 2024

(30) Foreign Application Priority Data
Jun. 21, 2022 (JP) 2022-099661
Jul. 26, 2022 (FR) 2207639

(51) Int. Cl. A61K 8/49; A61K 8/19; A61K 8/44; A61Q 19/02

(52) U.S. Cl. CPC A61K 8/4933; A61K 8/19; A61K 8/44; A61Q 19/02; A61K 2800/52

(57) ABSTRACT

A composition contains: (1) at least one thiopyridinone compound; (2) at least one pH adjusting agent, and (3) water. The pH of the composition is from 4.5 to 6.5. A composition including (1) thiopyridinone compound(s) can be provided with increased stability of the (1) thiopyridinone compound(s) over time, in particular even when the composition is maintained for a relatively long period of time under elevated temperature.

### TECHNICAL FIELD

The present invention relates to the stabilization of a thiopyridinone compound in a composition including the thiopyridinone compound.

### BACKGROUND ART

At various periods of their lives, some people see the appearance on their skin, and more in particular on their faces and hands, darker and/or more colored spots which give the skin heterogeneity. These spots are in particular due to a high concentration of melanin in the keratinocytes located at the surface of the skin.

The use of harmless topical depigmenting substances with good efficacy is most particularly desired for the purpose of treating pigmentation spots.

For example, arbutin, niacinamide and kojic acid are known as skin depigmenting agents.

On the other hand, WO2017/102349 discloses a new depigmenting or whitening agent, i.e., a thiopyridinone compound. The thiopyridinone compound can show strong depigmenting or whitening effects by reducing the production of melanin.

### DISCLOSURE OF INVENTION

However, it has been discovered that a thiopyridinone compound tends to be destabilized in a composition over time, in particular when the composition including the thiopyridinone compound is maintained for a relatively long period of time under elevated temperature.

Thus, an objective of the present invention is to provide a composition including thiopyridinone compound(s) with increased stability of the thiopyridinone compound(s) over time, in particular when the composition is maintained for a relatively long period of time under elevated temperature.

The above objective can be achieved by a composition comprising: (1) at least one compound selected from compounds of formula (I) below, tautomers of formula (I′) below, salts thereof, solvates, such as hydrates, thereof, optical isomers thereof, racemates thereof, and mixtures thereof:

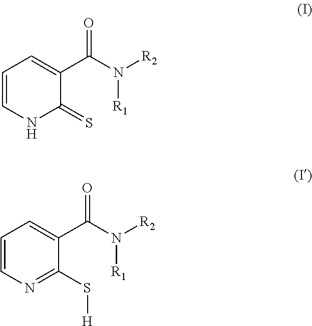

in which

R₁ denotes a radical chosen from
a) a hydrogen atom, and
b) a saturated, linear C₁-C₁₀ or branched C₃-C₁₀ alkyl group optionally substituted with one or more groups, which may be identical or different, chosen from
   i) —O—R₃, and
   ii) —S—R₃,

and

R₂ denotes a radical chosen from
a) a hydrogen atom,
b) a saturated, linear C₁-C₁₂ or branched C₃-C₁₂ or cyclic C₃-C₈ hydrocarbonated group optionally substituted with one or more groups, which may be identical or different, chosen from
   i) —O—R₃,
   ii) —S—R₃,
   iii) —C(O)—O—R₃, and
   iv) a C₅-C₁₂ aryl group optionally substituted with one or more hydroxyls and/or with one or more C₁-C₈ alkoxy radicals, and
c) a C₅-C₁₂ aryl group optionally substituted with one or more hydroxyls and/or with one or more C₁-C₈ alkoxy radicals

wherein

R₃ denotes a radical chosen from
a) a hydrogen atom, and
b) a saturated, linear C₁-C₁₀ or branched C₃-C₁₀ alkyl group;
   (2) at least one pH adjusting agent; and
   (3) water,

wherein

the pH of the composition is from 4.5 to 6.5, and preferably from 5 to 6.

It may be preferable that:

R₁ of formula (I) and (I′) represents a hydrogen atom;

or

R₁ of formula (I) and (I′) represents a linear (C₁-C₁₀) alkyl group or a branched (C₃-C₁₀) alkyl group, especially a linear (C₁-C₆) alkyl group or a branched (C₃-C₆) alkyl group, such as methyl, ethyl, n-pentyl, n-nonyl, isobutyl, more preferably ethyl, particularly the said alkyl group of R₁ is not substituted.

It may be preferable that:

R₂ of formula (I) and (I′) represents a hydrogen atom;

or

R₂ of formula (I) and (I′) represents a linear (C₁-C₁₀) alkyl group or a branched (C₃-C₁₀) alkyl group, especially a linear (C₁-C₆) alkyl group or a branched (C₃-C₆) alkyl group, such as methyl, ethyl, n-pentyl, n-nonyl, isobutyl, more preferably methyl or ethyl group; the said alkyl group of R₂ being not substituted.

It may be preferable that:

R₂ of formula (I) and (I′) represents a linear (C₁-C₁₀) alkyl group or a branched (C₃-C₁₀) alkyl group, especially a linear (C₁-C₆) alkyl group or a branched (C₃-C₆) alkyl group, such as methyl, ethyl, n-pentyl, n-nonyl, isobutyl, more preferably methyl or ethyl; the said alkyl group being substituted by one or more groups selected from i), ii), iii) and iv) as defined above, preferably the said alkyl group being substituted by one or two groups selected from i), ii) and iii), more preferably by one or two groups selected from i) and iii), better substituted by one group iii) as carboxy.

It may be preferable that:

R₂ of formula (I) and (I′) represents a (C₃-C₈) cycloalkyl group, preferably a (C₅-C₇) cycloalkyl group such cyclohexyl;

or

R₂ of formula (I) and (I′) represents a C₅-C₁₂ aryl group optionally substituted with one or more hydroxyls and/or with one or more C₁-C₈ alkoxy radicals, preferably a phenyl group particularly not substituted.

It may be preferable that:

R₃ of formula (I) and (I′) represents a hydrogen atom; or

R₃ of formula (I) and (I′) represents a saturated, linear C₁-C₁₀ or branched C₃-C₁₀ alkyl group; particularly a linear (C₁-C₆) alkyl group or a branched (C₃-C₆) alkyl group, preferably a (C₁-C₄) alkyl group such as methyl group.

It may be more preferable that:

R₁ of formula (I) and (I′) represents a radical chosen from
a) a hydrogen atom, and
b) a saturated, linear C₁-C₆ or branched C₃-C₆ alkyl group optionally substituted with one or more groups, which may be identical or different, chosen from
   i) —O—R₃, and
   ii) —S—R₃,
   preferably optionally substituted with one or more groups i);

R₂ of formula (I) and (I′) represents a radical chosen from
a) a hydrogen atom, and
b) a saturated, linear C₁-C₁₀ or branched C₃-C₁₀ or cyclic C₃-C₈ such as C₅-C₆ hydrocarbonated group, optionally substituted with one or more groups, which may be identical or different, chosen from
   i) —O—R₃,
   ii) —S—R₃,
   iii) —C(O)—O—R₃, and
   iv) a phenyl group optionally substituted with one or more hydroxyls and/or with one or more C₁-C₄ alkoxy radicals such as methoxy, preferably substituted with one or more groups selected from i) and iii), preferably iii) such as carboxy; and

R₃ of formula (I) and (I′) represents a radical chosen from
a) a hydrogen atom, and
b) a saturated, linear C₁-C₆ or branched C₃-C₆ alkyl group,

preferentially, the compounds of formula (I) and tautomer (I′), salts thereof, solvates, such as hydrates, thereof, optical isomers thereof, racemates thereof, and mixtures thereof, have the following meanings:

R₁ of formula (I) and (I′) represents a radical chosen from:
a) a hydrogen atom, and
b) a saturated, linear C₁-C₄ or branched C₃-C₄ alkyl group optionally substituted with one or more groups, which may be identical or different, chosen from i) —OR₃, more preferably not substituted;

R₂ of formula (I) and (I′) represents a radical chosen from
a) a hydrogen atom, and
b) a saturated, linear C₁-C₁₀ or branched C₃-C₁₀ or cyclic C₃-C₈ such as C₅-C₆ hydrocarbonated group, optionally substituted with one or more groups, which may be identical or different, chosen from
   i) —O—R₃,
   iii) —C(O)—O—R₃, and
   iv) a C₅-C₁₂ aryl group optionally substituted with one or more hydroxyls and/or with one or more C₁-C₄ alkoxy radicals; and

R₃ of formula (I) and (I′) represents a radical chosen from:
a) a hydrogen atom;
b) a saturated, linear C₁-C₄ or branched C₃-C₄ alkyl group such as methyl or ethyl.

The (1) compound may be selected from the compounds 1 to 24 below, tautomers thereof, salts thereof, solvates, such as hydrates, thereof, optical isomers thereof, racemates thereof, and mixtures thereof, particularly the compounds 1, 2, 4, 6, 7, 9, 11, 12, 14, 15, 16, 17, 18, 19, 20, or 21, more particularly 1, 9, 16, 18, 19, 20, or 21, preferably 18, 19, 20, or 21, and more preferably 20:

| No. | Structure | ChemicalName |
|---|---|---|
| 1 | 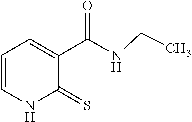 | N-ethyl-2-thioxo-1,2- dihydropyridine-3-carboxamide |
| 2 | 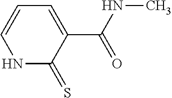 | N-methyl-2-thioxo-1,2- dihydropyridine-3-carboxamide |
| 3 | 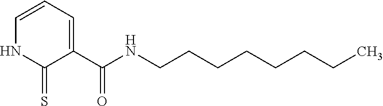 [SMILES: CCCCCCCCNC(=O)c1ccc[nH]c1=S] | N-octyl-2-thioxo-1,2- dihydropyridine-3-carboxamide |
| 4 | 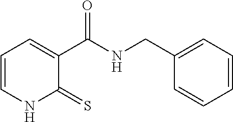 [SMILES: O=C(NCc1ccccc1)c1ccc[nH]c1=S] | N-benzyl-2-thioxo-1,2- dihydropyridine-3-carboxamide |
| 5 | 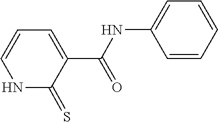 [SMILES: O=C(Nc1ccccc1)c1ccc[nH]c1=S] | N-phenyl-2-thioxo-1,2- dihydropyridine-3-carboxamide |
| 6 | 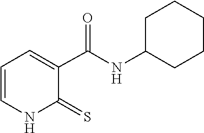 [SMILES: O=C(NC1CCCCC1)c1ccc[nH]c1=S] | N-cyclohexyl-2-thioxo-1,2- dihydropyridine-3-carboxamide |
| 7 | 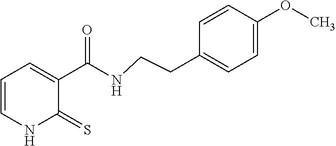 [SMILES: COc1ccc(CCNC(=O)c2ccc[nH]c2=S)cc1] | N-[2-(4-methoxyphenyl)ethyl]- 2-thioxo-1,2-dihydropyridine-3- carboxamide |
| 8 | 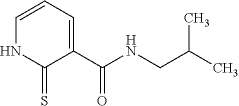 | N-(2-methylpropyl)-2-thioxo- 1,2-dihydropyridine-3- carboxamide |
| 9 | 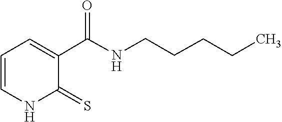 | N-pentyl-2-thioxo-1,2- dihydropyridine-3-carboxamide |
| 10 | 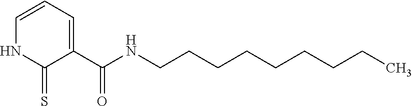 [SMILES: CCCCCCCCCNC(=O)c1ccc[nH]c1=S] | N-nonyl-2-thioxo-1,2- dihydropyridine-3-carboxamide |
| 11 | 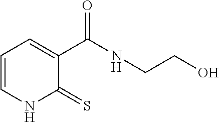 [SMILES: O=C(NCCO)c1ccc[nH]c1=S] | N-(2-hydroxyethyl)-2-thioxo- 1,2-dihydropyridine-3- carboxamide |
| 12 | 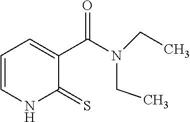 | N,N-diethyl 2- mercaptonicotinamide |
| 13 | 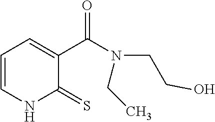 | N-ethyl-N-(2-hydroxyethyl)-2- thioxo-1,2-dihydropyridine-3- carboxamide |
| 14 | 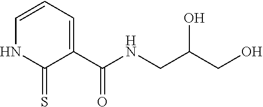 [SMILES: O=C(NCC(O)CO)c1ccc[nH]c1=S] | N-(2,3-dihydroxypropyl)-2- thioxo-1,2-dihydropyridine-3- carboxamide |
| 15 | 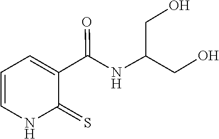 [SMILES: O=C(NC(CO)CO)c1ccc[nH]c1=S] | N-(1,3-dihydroxypropan-2-yl)- 2-thioxo-1,2-dihydropyridine-3- carboxamide |
| 16 | 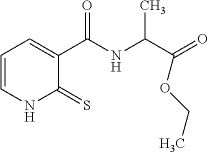 [SMILES: CCOC(=O)C(C)NC(=O)c1ccc[nH]c1=S] | Ethyl N-[(2-thioxo-1,2- dihydropyridin-3- yl)carbonyl]alaninate |
| 17 | 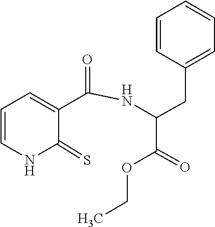 [SMILES: CCOC(=O)C(Cc1ccccc1)NC(=O)c1ccc[nH]c1=S] | Ethyl N-[(2-thioxo-1,2- dihydropyridin-3- yl)carbonyl]phenylalaninate |
| 18 | 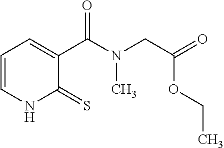 [SMILES: CCOC(=O)CN(C)C(=O)c1ccc[nH]c1=S] | EthylN-methyl-N-[(2-thioxo- 1,2-dihydropyridin-3- yl)carbonyl]glycinate |
| 19 | 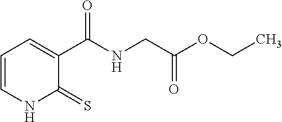 [SMILES: CCOC(=O)CNC(=O)c1ccc[nH]c1=S] | EthylN-[(2-thioxo-1,2- dihydropyridin-3- yl)carbonyl]glycinate |
| 20 | 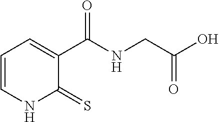 [SMILES: O=C(O)CNC(=O)c1ccc[nH]c1=S] | N-[(2-thioxo-1,2- dihydropyridin-3- yl)carbonyl]glycine |
| 21 | 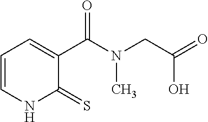 | N-methyl-N-[(2-thioxo-1,2- dihydropyridin-3- yl)carbonyl]glycine |
| 22 | 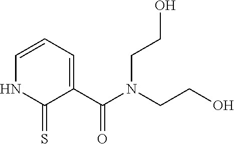 [SMILES: O=C(c1ccc[nH]c1=S)N(CCO)CCO] | N,N-bis(2-hydroxyethyl)-2- thioxo-1,2-dihydropyridine-3- carboxamide |
| 23 | 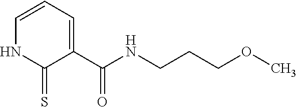 | N-(3-methoxypropyl)-2-thioxo- 1,2-dihydropyridine-3- carboxamide |
| 24 | 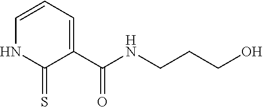 [SMILES: O=C(NCCCO)c1ccc[nH]c1=S] | N-butyl-2-thioxo-1,2- dihydropyridine-3-carboxamide |

The amount of the (1) compound(s) in the composition according to the present invention may be from 0.01% to 10% by weight, preferably from 0.05% to 5% by weight, and more preferably from 0.1% to 3% by weight, relative to the total weight of the composition.

The (2) pH adjusting agent may be selected from acidifying agents, basifying agents, and mixtures thereof.

The amount of the (2) pH adjusting agent(s) in the composition according to the present invention may be from 0.01% to 15% by weight, preferably from 0.05% to 10% by weight, more preferably from 0.1% to 5% by weight, relative to the total weight of the composition.

The amount of the (3) water in the composition according to the present invention may be from 20% to 99% by weight, preferably from 30% to 95% by weight, more preferably from 40% to 90% by weight, relative to the total weight of the composition.

The composition according to the present invention may be for whitening a keratin substance, preferably skin.

The present invention also relates to a cosmetic process, preferably a whitening process, for a keratin substance, preferably skin, comprising the step of: applying to the keratin substance the composition according to the present invention.

Another aspect of the present invention is a use of (2) at least one pH adjusting agent in a composition comprising (1) at least one compound selected from compounds of formula (I) below, tautomers of formula (I′) below, salts thereof, solvates, such as hydrates, thereof, optical isomers thereof, racemates thereof, and mixtures thereof:

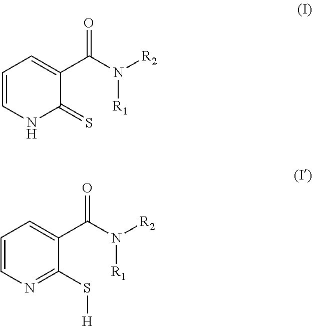

in which

R₁ denotes a radical chosen from
a) a hydrogen atom, and
b) a saturated, linear C₁-C₁₀ or branched C₃-C₁₀ alkyl group optionally substituted with one or more groups, which may be identical or different, chosen from
   i) —O—R₃, and
   ii) —S—R₃,

and

R₂ denotes a radical chosen from
a) a hydrogen atom,
b) a saturated, linear C₁-C₁₂ or branched C₃-C₁₂ or cyclic C₃-C₈ hydrocarbonated group optionally substituted with one or more groups, which may be identical or different, chosen from
   i) —O—R₃,
   ii) —S—R₃,
   iii) —C(O)—O—R₃, and
   iv) a C₅-C₁₂ aryl group optionally substituted with one or more hydroxyls and/or with one or more C₁-C₈ alkoxy radicals, and
c) a C₅-C₁₂ aryl group optionally substituted with one or more hydroxyls and/or with one or more C₁-C₈ alkoxy radicals

wherein

R₃ denotes a radical chosen from
a) a hydrogen atom, and
b) a saturated, linear C₁-C₁₀ or branched C₃-C₁₀ alkyl group

in order to control the pH of the composition to be from 4.5 to 6.5 for stabilizing the (1) compound(s).

### BEST MODE FOR CARRYING OUT THE INVENTION

After diligent research, the inventors have discovered that it is possible to provide a composition including a thiopyridinone compound or thiopyridinone compounds with increased stability of the thiopyridinone compound(s) over time, in particular even when the composition is maintained for a relatively long period of time under elevated temperature.

Thus, the composition according to the present invention comprises: (1) at least one compound selected from compounds of formula (I) below, tautomers of formula (I′) below, salts thereof, solvates, such as hydrates, thereof, optical isomers thereof, racemates thereof, and mixtures thereof (hereafter, the compound is referred to as “thiopyridinone compound”):

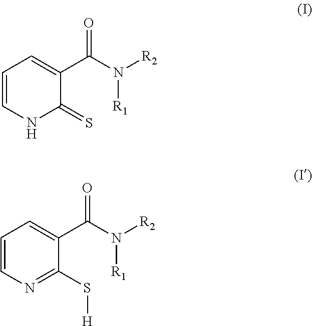

in which

R₁ denotes a radical chosen from
a) a hydrogen atom, and
b) a saturated, linear C₁-C₁₀ or branched C₃-C₁₀ alkyl group optionally substituted with one or more groups, which may be identical or different, chosen from
   i) —O—R₃, and
   ii) —S—R₃,

and

R₂ denotes a radical chosen from
a) a hydrogen atom,
b) a saturated, linear C₁-C₁₂ or branched C₃-C₁₂ or cyclic C₃-C₈ hydrocarbonated group optionally substituted with one or more groups, which may be identical or different, chosen from
   i) —O—R₃,
   ii) —S—R₃,
   iii) —C(O)—O—R₃, and
   iv) a C₅-C₁₂ aryl group optionally substituted with one or more hydroxyls and/or with one or more C₁-C₈ alkoxy radicals, and
c) a C₅-C₁₂ aryl group optionally substituted with one or more hydroxyls and/or with one or more C₁-C₈ alkoxy radicals

wherein

R₃ denotes a radical chosen from
a) a hydrogen atom, and
b) a saturated, linear C₁-C₁₀ or branched C₃-C₁₀ alkyl group;

(2) at least one pH adjusting agent; and

(3) water,

wherein

the pH of the composition is from 4.5 to 6.5, and preferably from 5 to 6.

The composition according to the present invention can show increased stability of the (1) thiopyridinone compound therein.

In other words, the composition according to the present invention can increase the stability of the (1) thiopyridinone compound therein. The term “stability” of the (1) thiopyridinone compound can be determined by the change in the amount of the (1) thiopyridinone compound in the composition according to the present invention during a certain period of time. An increased “stability” means that the change in the amount of the (1) thiopyridinone compound over time is more limited.

The composition according to the present invention can show increased stability of the (1) thiopyridinone compound therein, even when the composition is maintained for a relatively long period of time such as two weeks under elevated temperature such as 55° C.

Therefore, the composition according to the present invention can be stored for a long period of time even under hot conditions.

In addition, the increased stability of the (1) thiopyridinone compound can provide improved or enhanced bioavailability of the (1) thiopyridinone compound which can function as a depigmenting or whitening agent. Therefore, the composition according to the present invention can provide enhanced or improved depigmenting or whitening effects.

Hereafter, the composition, use and the like according to the present invention will be described in a detailed manner.

### Composition

The composition according to the present invention comprises:

(1) at least one thiopyridinone compound;

(2) at least one pH adjusting agent; and

(3) water,

and

the pH of the composition is from 4.5 to 6.5.

The (1) thiopyridinone compound, the (2) pH adjusting agent, and the (3) water, as well as the other features of the composition according to the present invention will be explained below.

### Thiopyridinone Compound

The composition according to the present invention comprises (1) at least one thiopyridinone compound. Two or more (1) thiopyridinone compounds may be used in combination. Thus, a single type of (1) thiopyridinone compound or a combination of different types of (1) thiopyridinone compounds may be used.

The (1) thiopyridinone compound is selected from compounds of formula (I) below, tautomers of formula (I′) below, salts thereof, solvates, such as hydrates, thereof, optical isomers thereof, racemates thereof, and mixtures thereof:

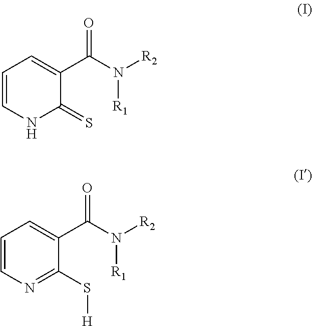

in which

R₁ denotes a radical chosen from
a) a hydrogen atom, and
b) a saturated, linear C₁-C₁₀ or branched C₃-C₁₀ alkyl group optionally substituted with one or more groups, which may be identical or different, chosen from
   i) —O—R₃, and
   ii) —S—R₃,

and

R₂ denotes a radical chosen from
a) a hydrogen atom,
b) a saturated, linear C₁-C₁₂ or branched C₃-C₁₂ or cyclic C₃-C₈ hydrocarbonated group optionally substituted with one or more groups, which may be identical or different, chosen from
   i) —O—R₃,
   ii) —S—R₃,
   iii) —C(O)—O—R₃, and
   iv) a C₅-C₁₂ aryl group optionally substituted with one or more hydroxyls and/or with one or more C₁-C₈ alkoxy radicals, and
c) a C₅-C₁₂ aryl group optionally substituted with one or more hydroxyls and/or with one or more C₁-C₈ alkoxy radicals

wherein

R₃ denotes a radical chosen from
a) a hydrogen atom, and
b) a saturated, linear C₁-C₁₀ or branched C₃-C₁₀ alkyl group.

Hereafter, for the purposes of the present invention and unless otherwise indicated:
a “saturated, linear C₁-C₁₂ or branched C₃-C₁₂” hydrocarbonated group is equivalent to a “linear (C₁-C₁₂) alkyl or branched (C₃-C₁₂) alkyl group” which corresponds to a saturated, linear C₁-C₁₂ or branched C₃-C₁₂ hydrocarbon-based group, preferably a linear C₁-C₁₀ or branched C₃-C₁₀ hydrocarbon-based group, and more preferably a linear C₁-C₆ or branched C₃-C₆ hydrocarbon-based group; Preferentially, the linear or branched groups may be chosen from methyl, ethyl, propyl, isopropyl, butyl, isobutyl and tert-butyl, pentyl, hexyl, heptyl, octyl, nonyl and decyl; More preferentially, the saturated linear or branched alkyl groups may be chosen from methyl, ethyl, propyl, isopropyl, butyl, isobutyl and tert-butyl, pentyl, hexyl, heptyl and octyl, such as methyl, ethyl, n-pentyl, n-nonyl, isobutyl;
a saturated “cyclic C₃-C₈” hydrocarbonated group is a mono or bicyclic cycloalkyl group containing from 3 to 8 carbon atoms, and especially is a monocyclic cycloalkyl group in C₅ to C₇ such as a cyclohexyl group,
an “alkoxy radical” is an alkyl-oxy radical for which the alkyl radical is a linear or branched C₁-C₁₆, and preferentially a C₁-C₈ hydrocarbon-based radical;
when the alkoxy group is optionally substituted, this implies that the alkyl group is optionally substituted as defined above;
an “aryl” group represents a fused or non-fused monocyclic or bicyclic carbon-based group comprising from 5 to 12 carbon atoms, preferably from 6 to 10 carbon atoms, and in which at least one ring is aromatic; preferentially, the aryl radical is a phenyl, biphenyl, naphthyl, more preferably a phenyl group;
the term “at least one” is equivalent to the term “one or more”; and
the term “inclusive” for a range of concentrations means that the limits of that range are included in the defined range.

The salts of the compounds of formula (I), (I′), (II), or (II') as defined below comprise the conventional non-toxic salts of said compounds, such as those formed from organic or inorganic acid or from organic or inorganic base.

As salts of the compounds of formula (I), (I′), (II), or (II′), mention may be made of: the salts obtained by addition of the compound of formula (I) or (II) to:
a mineral base, such as sodium hydroxide, potassium hydroxide, calcium hydroxide, ammonium hydroxide, magnesium hydroxide, lithium hydroxide, and sodium, potassium or calcium carbonate or hydrogen carbonate for example;
   or
an organic base such as a primary, secondary or tertiary alkylamine, for example triethylamine or butylamine. This primary, secondary or tertiary alkylamine may comprise one or more nitrogen and/or oxygen atoms and may thus comprise, for example, one or more alcohol functions. Mention may be made in particular of 2-amino-2-methylpropanol, ethanolamine, triethanolamine, 2-dimethylaminopropanol, 2-amino-2-(hydroxymethyl)-1,3-propanediol and 3-(dimethylamino)propylamine.

Mention may also be made of the salts of amino acids, for instance lysine, arginine, guanidine, glutamic acid and aspartic acid. Advantageously, the salts of the compounds of formula (I) or (II) (when it comprises a carboxy group) may be chosen from alkali metal or alkaline-earth metal salts such as sodium, potassium, calcium or magnesium salts and ammonium salts.

As “organic or inorganic acid salt” is more particularly chosen from salts chosen from a salt derived from i) hydrochloric acid HCl, ii) hydrobromic acid HBr, iii) sulfuric acid H₂SO₄, iv) alkylsulfonic acids: Alk-S(O)₂OH such as methanesulfonic acid and ethanesulfonic acid; v) arylsulfonic acids: Ar—S(O)₂OH such as benzenesulfonic acid and toluenesulfonic acid; vi) citric acid; vii) succinic acid; viii) tartaric acid; ix) lactic acid; x) alkoxysulfinic acids: Alk-O—S(O)OH such as methoxysulfinic acid and ethoxysulfinic acid; xi) aryloxysulfinic acids such as tolueneoxysulfinic acid and phenoxysulfinic acid; xii) phosphoric acid H₃PO_{4;} xiii) acetic acid CH₃C(O)OH; xiv) triflic acid CF₃SO₃H; and xv) tetrafluoroboric acid HBF₄.

The acceptable solvates of the compounds described in the specification comprise conventional solvates such as those formed during the preparation of said compounds owing to the presence of solvents. Mention may be made, by way of example, of the solvates due to the presence of water or of linear or branched alcohols, such as ethanol or isopropanol.

The optical isomers are in particular, the enantiomers and the diastereoisomers.

Compound (I′) is the tautomer form of compound (I) when a tautomeric equilibrium exists according to the following scheme:

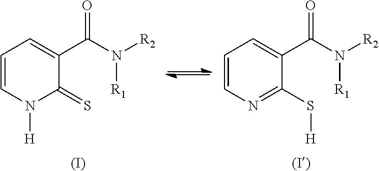

According to one embodiment of the present invention, R₁ represents one hydrogen atom.

According to another embodiment of the present invention, R₁ represents a linear (C₁-C₁₀) alkyl group or a branched (C₃-C₁₀) alkyl group, especially a linear (C₁-C₆) alkyl group or a branched (C₃-C₆) alkyl group, such as methyl, ethyl, n-pentyl, n-nonyl, isobutyl, more preferably ethyl. Particularly the said alkyl group of R₁ is not substituted.

According to one embodiment of the present invention, R₂ represents one hydrogen atom.

According to another embodiment of the present invention, R₂ represents a linear (C₁-C₁₀) alkyl group or a branched (C₃-C₁₀) alkyl group, especially a linear (C₁-C₆) alkyl group or a branched (C₃-C₆) alkyl group, such as methyl, ethyl, n-pentyl, n-nonyl, isobutyl, more preferably methyl or ethyl group; the said alkyl group of R₂ being not substituted.

According to another embodiment of the present invention, R₂ represents a linear (C₁-C₁₀) alkyl group or a branched (C₃-C₁₀) alkyl group, especially a linear (C₁-C₆) alkyl group or a branched (C₃-C₆) alkyl group, such as methyl, ethyl, n-pentyl, n-nonyl, isobutyl, more preferably methyl or ethyl; the said alkyl group being substituted by one or more groups selected from i), ii), iii) and iv) as defined above. Preferably the said alkyl group being substituted by one or two groups selected from i), ii), and iii), more preferably by one or two groups selected from i) and iii), better substituted by one group iii) as carboxy.

Another variant for radical R₂ is that the said alkyl group being substituted by one group iv) especially substituted by one phenyl group.

According to another embodiment of the present invention, R₂ represents a (C₃-C₈) cycloalkyl group, preferably a (C₅-C₇) cycloalkyl group such cyclohexyl.

According to another embodiment of the present invention, R₂ represents a C₅-C₁₂ aryl group optionally substituted with one or more hydroxyls and/or with one or more C₁-C₈ alkoxy radicals, preferably a phenyl group particularly not substituted.

According to an embodiment, R₃ represents a hydrogen atom.

According to another embodiment, R₃ represents a saturated, linear C₁-C₁₀ or branched C3-C10 alkyl group; particularly a linear (C₁-C₆) alkyl group or a branched (C₃-C₆) alkyl group, preferably (C₁-C₄) alkyl group such as methyl group.

Preferably, the compounds of formula (I) and tautomer (I′) or their salts, their optical isomers, racemates, and/or solvates such as hydrates and the thereof, alone or as a mixture have the following meanings:

R₁ denotes a radical chosen from
a) a hydrogen atom, and
b) a saturated, linear C₁-C₆ or branched C₃-C₆ alkyl group optionally substituted with one or more groups, which may be identical or different, chosen from
   i) —O—R₃, and
   ii) —S—R₃, preferably optionally substituted with one or more groups i);

R₂ denotes a radical chosen from
a) a hydrogen atom;
b) a saturated, linear C₁-C₁₀ or branched C₃-C₁₀ or cyclic C₃-C₈ such as C₅-C₆ hydrocarbonated group optionally substituted with one or more groups, which may be identical or different, chosen from:
   i) —O—R₃,
   ii) —S—R₃,
   iii) —C(O)—O—R₃, and
   iv) a phenyl group optionally substituted with one or more hydroxyls and/or with one or more C₁-C₄ alkoxy radicals such as methoxy, preferably substituted with one or more groups selected from i) and iii), preferably iii) such as carboxy; and

R₃ denotes a radical chosen from
a) a hydrogen atom, and
b) a saturated, linear C₁-C₆ or branched C₃-C₆ alkyl group

Preferentially, the compounds of formula (I) and tautomer (I′) or their salts, their optical isomers, racemates, and/or solvates such as hydrates and the thereof, alone or as a mixture have the following meanings:

R₁ denotes a radical chosen from
a) a hydrogen atom, and
b) a saturated, linear C₁-C₄ or branched C₃-C₄ alkyl group optionally substituted with one or more groups, which may be identical or different, chosen from i) —OR₃, more preferably not substituted;

R₂ denotes a radical chosen from
a) a hydrogen atom; and
b) a saturated, linear C₁-C₁₀ or branched C₃-C₁₀ or cyclic C₃-C₈ such as C₅-C₆ hydrocarbonated group, optionally substituted with one or more groups, which may be identical or different, chosen from
   i) —O—R₃,
   iii) —C(O)—O—R₃,
   iv) a C₅-C₁₂ aryl group optionally substituted with one or more hydroxyls and/or with one or more C₁-C₄ alkoxy radicals; and

R₃ denotes a radical chosen from
a) a hydrogen atom, and
b) a saturated linear C₁-C₄ or branched C₃-C₄ alkyl group such as methyl or ethyl.

Preferentially, the compounds of formula (I) and tautomer (I′) or their salts, their optical isomers, racemates, and/or solvates such as hydrates and the thereof, alone or as a mixture have the following meanings:

R₁ is a hydrogen atom; and

R₂ denotes a radical chosen from
a) a hydrogen atom, and
b) a saturated, linear C₁-C₅ or branched C₃-C₅ or cyclic C₃-C₈ such as C₅-C₆ hydrocarbonated group, substituted with one or more groups, which may be identical or different, chosen from v) —C(O)—O—R₃, preferably substituted with one group iii) —C(O)—O—R₃; R₂ is even more preferably a saturated, linear C₁-C₄ or branched C₃-C₄ hydrocarbonated group substituted with one group iii) —C(O)—OR₃; and

R₃ denotes a radical chosen from
a) a hydrogen atom, and
b) a saturated linear C₁-C₄ or branched C₃-C₄ alkyl group such as methyl or ethyl.

According to another preferred embodiment, compounds of formula (I) and tautomer (I′) are selected among the compounds of formula (II) below and also the tautomers thereof of formula (II′) below, the salts thereof, the solvates thereof and the optical isomers thereof, and the racemates thereof, alone or as a mixture:

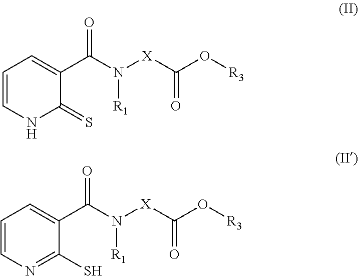

In formula (II) and (II′), R₁ and R₃ have the same meaning as R₁ and R₃ in the compounds of formula (I) and (I′) and X denotes an alkylene radical —(CH₂)ₙ— with n being an integer ranging inclusively from 1 to 10, preferably ranging from 1 to 6, more preferably ranging from 1 to 4, such as 1, preferably R₃ represents a hydrogen atom.

Among the compounds of formula (I), the following compounds are preferably used and their tautomer or their salts, their optical isomers, racemates, and/or solvates such as hydrates and the thereof, alone or as a mixture:

| No. | Structure | Chemicalname | CAS No. |
|---|---|---|---|
| 1 | 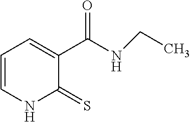 | N-ethyl-2-thioxo-1,2- dihydropyridine-3- carboxamide | 91859-75-5 |
| 2 | 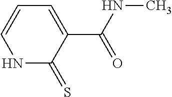 | N-methyl-2-thioxo- 1,2-dihydropyridine-3- carboxamide | 91859-74-4 |
| 3 | 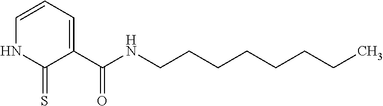 [SMILES: CCCCCCCCNC(=O)c1ccc[nH]c1=S] | N-octyl-2-thioxo-1,2- dihydropyridine-3- carboxamide | 91859-77-7 |
| 4 | 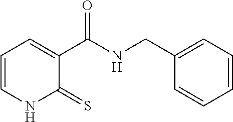 [SMILES: O=C(NCc1ccccc1)c1ccc[nH]c1=S] | N-benzyl-2-thioxo-1,2- dihydropyridine-3- carboxamide | 91859-79-9 |
| 5 | 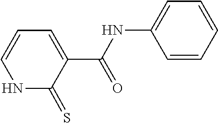 [SMILES: O=C(Nc1ccccc1)c1ccc[nH]c1=S] | N-phenyl-2-thioxo- 1,2-dihydropyridine-3- carboxamide | 104857-16-1 |
| 6 | 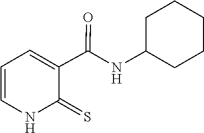 [SMILES: O=C(NC1CCCCC1)c1ccc[nH]c1=S] | N-cyclohexyl-2- thioxo-1,2- dihydropyridine-3- carboxamide | 91859-78-8 |
| 7 | 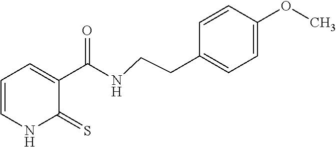 [SMILES: COc1ccc(CCNC(=O)c2ccc[nH]c2=S)cc1] | N-[2-(4- methoxyphenyl)ethyl]- 2-thioxo-1,2- dihydropyridine-3- carboxamide | 923682-88-6 |
| 8 | 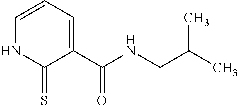 | N-(2-methylpropyl)-2- thioxo-1,2- dihydropyridine-3- carboxamide | 1100027-79-9 |
| 9 | 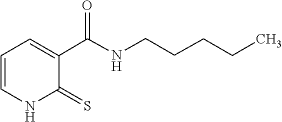 | N-pentyl-2-thioxo-1,2- dihydropyridine-3- carboxamide | 330667-57-7 |
| 10 | 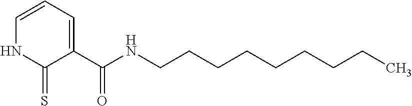 [SMILES: CCCCCCCCCNC(=O)c1ccc[nH]c1=S] | N-nonyl-2-thioxo-1,2- dihydropyridine-3- carboxamide | 1031149-44-6 |
| 11 | 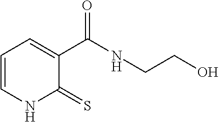 [SMILES: O=C(NCCO)c1ccc[nH]c1=S] | N-(2-hydroxyethyl)-2- thioxo-1,2- dihydropyridine-3- carboxamide | |
| 12 | 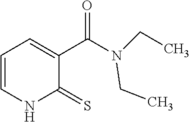 | N,N-diethyl2- mercaptonicotinamide | |
| 13 | 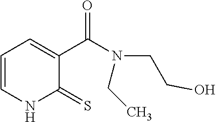 | N-ethyl-N-(2- hydroxyethyl)-2- thioxo-1,2- dihydropyridine-3- carboxamide | |
| 14 | 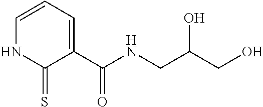 [SMILES: O=C(NCC(O)CO)c1ccc[nH]c1=S] | N-(2,3- dihydroxypropyl)-2- thioxo-1,2- dihydropyridine-3- carboxamide | |
| 15 | 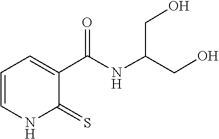 [SMILES: O=C(NC(CO)CO)c1ccc[nH]c1=S] | N-(1,3- dihydroxypropan-2- yl)-2-thioxo-1,2- dihydropyridine-3- carboxamide | |
| 16 | 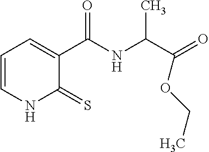 [SMILES: CCOC(=O)C(C)NC(=O)c1ccc[nH]c1=S] | EthylN-[(2-thioxo- 1,2-dihydropyridin-3- yl)carbonyl]alaninate | |
| 17 | 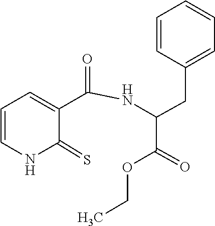 [SMILES: CCOC(=O)C(Cc1ccccc1)NC(=O)c1ccc[nH]c1=S] | EthylN-[(2-thioxo- 1,2-dihydropyridin-3- yl)carbonyl]phenyl alaninate | |
| 18 | 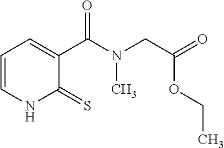 [SMILES: CCOC(=O)CN(C)C(=O)c1ccc[nH]c1=S] | EthylN-methyl-N-[(2- thioxo-1,2- dihydropyridin-3- yl)carbonyl]glycinate | |
| 19 | 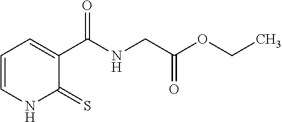 [SMILES: CCOC(=O)CNC(=O)c1ccc[nH]c1=S] | EthylN-[(2-thioxo- 1,2-dihydropyridin-3- yl)carbonyl]glycinate | |
| 20 | 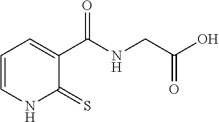 [SMILES: O=C(O)CNC(=O)c1ccc[nH]c1=S] | N-[(2-thioxo-1,2- dihydropyridin-3- yl)carbonyl]glycine | |
| 21 | 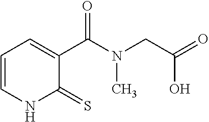 | N-methyl-N-[(2- thioxo-1,2- dihydropyridin-3- yl)carbonyl]glycine | |
| 22 | 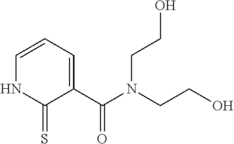 [SMILES: O=C(c1ccc[nH]c1=S)N(CCO)CCO] | N,N-bis(2- hydroxyethyl)-2- thioxo-1,2- dihydropyridine-3- carboxamide | |
| 23 | 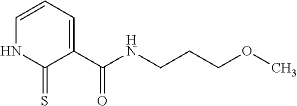 | N-(3-methoxypropyl)- 2-thioxo-1,2- dihydropyridine-3- carboxamide | |

| | | |
|---|---|---|
| 24 | 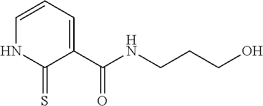 [SMILES: O=C(NCCCO)c1ccc[nH]c1=S] | N-butyl-2-thioxo-1,2- dihydropyridine-3- carboxamide |

Among these compounds, the following compounds are more particularly preferred:

| No. | Structure | Chemicalname | CAS No. |
|---|---|---|---|
| 1 | 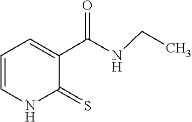 | N-ethyl-2-thioxo-1,2- dihydropyridine-3- carboxamide | 91859-75-5 |
| 2 | 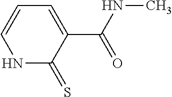 | N-methyl-2-thioxo- 1,2-dihydropyridine-3- carboxamide | 91859-74-4 |
| 4 | 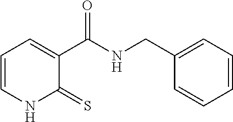 [SMILES: O=C(NCc1ccccc1)c1ccc[nH]c1=S] | N-benzyl-2-thioxo-1,2- dihydropyridine-3- carboxamide | 91859-79-9 |
| 6 | 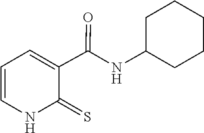 [SMILES: O=C(NC1CCCCC1)c1ccc[nH]c1=S] | N-cyclohexyl-2- thioxo-1,2- dihydropyridine-3- carboxamide | 91859-78-8 |
| 7 | 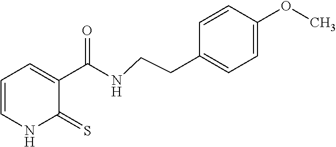 [SMILES: COc1ccc(CCNC(=O)c2ccc[nH]c2=S)cc1] | N-[2-(4- methoxyphenyl)ethyl]- 2-thioxo-1,2- dihydropyridine-3- carboxamide | 923682-88-6 |
| 9 | 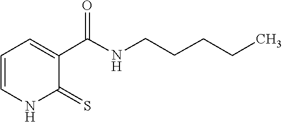 | N-pentyl-2-thioxo-1,2- dihydropyridine-3- carboxamide | 330667-57-7 |
| 11 | 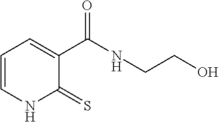 [SMILES: O=C(NCCO)c1ccc[nH]c1=S] | N-(2-hydroxyethyl)-2- thioxo-1,2- dihydropyridine-3- carboxamide | |
| 12 | 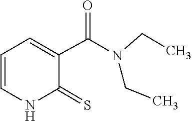 | N,N-diethyl2- mercaptonicotinamide | |
| 14 | 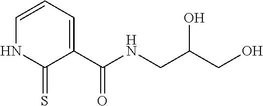 [SMILES: O=C(NCC(O)CO)c1ccc[nH]c1=S] | N-(2,3- dihydroxypropyl)-2- thioxo-1,2- dihydropyridine-3- carboxamide | |
| 15 | 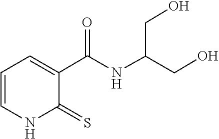 [SMILES: O=C(NC(CO)CO)c1ccc[nH]c1=S] | N-(1,3- dihydroxypropan-2- yl)-2-thioxo-1,2- dihydropyridine-3- carboxamide | |
| 16 | 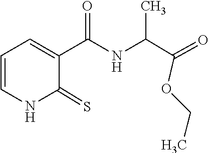 [SMILES: CCOC(=O)C(C)NC(=O)c1ccc[nH]c1=S] | Ethyl N-[(2-thioxo- 1,2-dihydropyridin-3- yl)carbonyl]alaninate | |
| 17 | 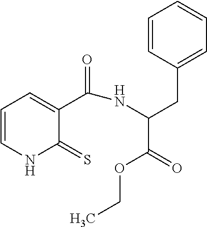 [SMILES: CCOC(=O)C(Cc1ccccc1)NC(=O)c1ccc[nH]c1=S] | Ethyl N-[(2-thioxo- 1,2-dihydropyridin-3- yl)carbonyl]phenyl alaninate | |
| 18 | 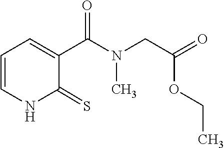 [SMILES: CCOC(=O)CN(C)C(=O)c1ccc[nH]c1=S] | Ethyl N-methyl-N-[(2- thioxo-1,2- dihydropyridin-3- yl)carbonyl]glycinate | |
| 19 | 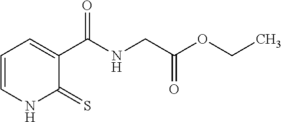 [SMILES: CCOC(=O)CNC(=O)c1ccc[nH]c1=S] | Ethyl N-[(2-thioxo- 1,2-dihydropyridin-3- yl)carbonyl]glycinate | |
| 20 | 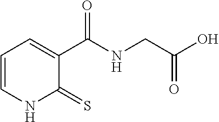 [SMILES: O=C(O)CNC(=O)c1ccc[nH]c1=S] | N-[(2-thioxo-1,2- dihydropyridin-3- yl)carbonyl]glycine | |
| 21 | 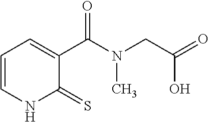 | N-methyl-N-[(2- thioxo-1,2- dihydropyridin-3- yl)carbonyl]glycine | |

More preferably, among these compounds, the following compounds are more particularly preferred:

| No. | Structure | Chemical name | CAS No. |
|---|---|---|---|
| 1 | 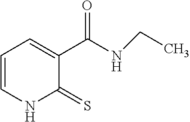 | N-ethyl-2-thioxo-1,2- dihydropyridine-3- carboxamide | 91859-75-5 |
| 9 | 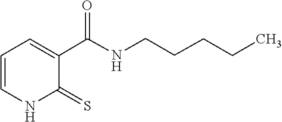 | N-pentyl-2-thioxo-1,2- dihydropyridine-3- carboxamide | 330667-57-7 |
| 16 | 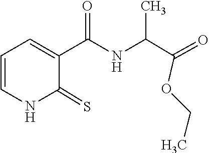 [SMILES: CCOC(=O)C(C)NC(=O)c1ccc[nH]c1=S] | EthylN-[(2-thioxo-1,2- dihydropyridin-3- yl)carbonyl]alaninate | |
| 18 | 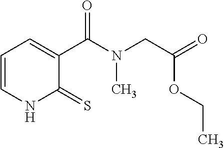 [SMILES: CCOC(=O)CN(C)C(=O)c1ccc[nH]c1=S] | EthylN-methyl-N-[(2- thioxo-1,2- dihydropyridin-3- yl)carbonyl]glycinate | |
| 19 | 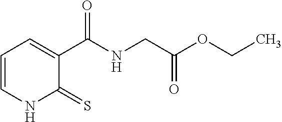 [SMILES: CCOC(=O)CNC(=O)c1ccc[nH]c1=S] | EthylN-[(2-thioxo-1,2- dihydropyridin-3- yl)carbonyl]glycinate | |
| 20 | 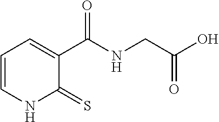 [SMILES: O=C(O)CNC(=O)c1ccc[nH]c1=S] | N-[(2-thioxo-1,2- dihydropyridin-3- yl)carbonyl]glycine | |
| 21 | 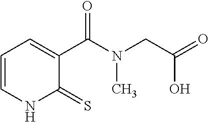 | N-methyl-N-[(2- thioxo-1,2- dihydropyridin-3- yl)carbonyl]glycine | |

Even more preferably, Among these compounds, the following compounds are more particularly preferred:

| | | Chemical | CAS |
|---|---|---|---|
| No. | Structure | name | No. |
| 18 | 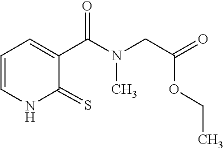 [SMILES: CCOC(=O)CN(C)C(=O)c1ccc[nH]c1=S] | Ethyl N-methyl- N-[(2- thioxo-1,2- dihydropyridin- 3- yl)carbonyl] glycinate | |
| 19 | 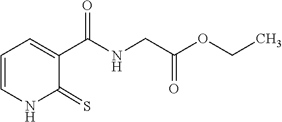 [SMILES: CCOC(=O)CNC(=O)c1ccc[nH]c1=S] | EthylN-[(2- thioxo-1,2- dihydropyridin- 3- yl)carbonyl] glycinate | |
| 20 | 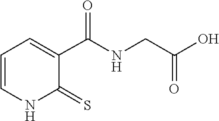 [SMILES: O=C(O)CNC(=O)c1ccc[nH]c1=S] | N-[(2- thioxo-1,2- dihydropyridin- 3- yl)carbonyl] glycine | |
| 21 | 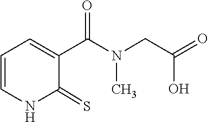 | N-methyl- N-[(2- thioxo-1,2- dihydropyridin- 3- yl)carbonyl] glycine | |

In the most preferred embodiment, the compound according to the present invention is the following:

| | | |
|---|---|---|
| 20 | 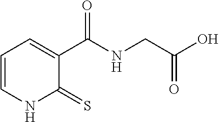 [SMILES: O=C(O)CNC(=O)c1ccc[nH]c1=S] | N-[(2-thioxo-1,2- dihydropyridin-3- yl)carbonyl]glycine |

All the compounds above can be obtained by a chemical method known by person man skilled in the art, from commercially available reagents.

The (1) thiopyridinone compound can be prepared in accordance with the process described in, for example, EP-A-3390363 or WO 2017/102349, which is herein incorporated by reference.

The (1) thiopyridinone compound may be an active ingredient or active compound in cosmetics or dermatological products. The term “active” ingredient or compound used herein means an ingredient or compound which has a cosmetic or dermatological active property, such as anti-oxidant, whitening, UV-filtering effects and anti-bacterial effects. The (1) thiopyridinone compound used in the present invention can function as a depigmenting, bleaching or whitening agent, and thus the composition according to the present invention may be used as a whitening product or as a cosmetic composition for a whitening keratin substance.

The (1) thiopyridinone compound may be used as an agent for depigmenting, bleaching or whitening the skin, body hairs, the eyelashes or head hair, and also the lips and/or the nails, and preferably the skin, in particular for eliminating pigmentation spots or senescence spots, and/or as an anti-tanning agent.

The amount of the (1) thiopyridinone compound(s) in the composition according to the present invention may be 0.01% by weight or more, preferably 0.05% by weight or more, and more preferably 0.1% by weight or more, relative to the total weight of the composition.

On the other hand, the amount of the (1) thiopyridinone compound(s) in the composition according to the present invention may be 10% by weight or less, preferably 5% by weight or less, and more preferably 3% by weight or less, relative to the total weight of the composition.

The amount of the (1) thiopyridinone compound(s) in the composition according to the present invention may range from 0.01% to 10% by weight, preferably from 0.05% to 5% by weight, more preferably from 0.1% to 3% by weight, relative to the total weight of the composition.

### pH Adjusting Agent

The composition according to the present invention comprises (2) at least one pH adjusting agent (pH adjuster). Two or more pH adjusting agents may be used in combination. Thus, a single type of pH adjusting agent or a combination of different types of pH adjusting agents may be used.

The (2) pH adjusting agent is different from the (1) thiopyridinone compound.

As the (2) pH adjusting agent, at least one acidifying agent and/or at least one basifying agent (alkaline agent) may be used.

The acidifying agent may be a monovalent or polyvalent, such as divalent, acid.

The acidifying agents can be, for example, mineral (inorganic) acids such as hydrochloric acid, sulfuric acid, phosphoric acid, or organic acids such as carboxylic acids, for instance tartaric acid, citric acid, and lactic acid, as well as sulphonic acids.

The basifying agent may be a monovalent or polyvalent, such as divalent, base.

The basifying agents may be mineral (inorganic) or organic, or hybrid.

The mineral basifying agents may be chosen from aqueous ammonia; alkali metal carbonates or bicarbonates such as sodium or potassium carbonates and sodium or potassium bicarbonates; alkali metal hydroxides such as sodium hydroxide and potassium hydroxide; and mixtures thereof.

The organic basifying agents may be chosen from organic amines with a pKb at 25° C. of less than 12, preferably less than 10, and even more advantageously less than 6. It should be noted that it is the pKb corresponding to the function of highest basicity. In addition, the organic amines do not comprise any alkyl or alkenyl fatty chains comprising more than ten carbon atoms.

The organic basifying agent may be chosen, for example, from alkanolamines, oxyethylenated and/or oxypropylenated ethylenediamines, amino acids and amine compounds of formula (III) below:

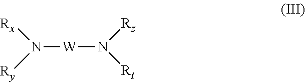

in which

W represents a C₁-C₆ divalent alkylene radical optionally substituted with one or more hydroxyl groups or a C₁-C₆ alkyl radical, and optionally interrupted with one or more heteroatoms such as O and N, and

Rₓ, R_{y}, R_{z}, and Rₜ, which may be identical or different, represent a hydrogen atom or a C₁-C₆ alkyl, C₁-C₆ hydroxyalkyl, or C₁-C₆ aminoalkyl radical.

Examples of the amine compounds of formula (III) that may be mentioned include 1,3-diaminopropane, 1,3-diamino-2-propanol, spermine and spermidine.

The term “alkanolamine” means an organic amine comprising a primary, secondary or tertiary amine function, and one or more linear or branched C₁-C₈ alkyl groups bearing one or more hydroxyl radicals.

Alkanolamines such as monoalkanolamines, dialkanolamines or trialkanolamines comprising one to three identical or different C₁-C₄ hydroxyalkyl radicals may be suitable for the present invention. Among the compounds of this type, mention may be made of monoethanolamine (MEA), diethanolamine, triethanolamine, monoisopropanolamine, diisopropanolamine, N-dimethylaminoethanolamine, 2-amino-2-methyl-1-propanol, triisopropanolamine, 2-amino-2-methyl-1,3-propanediol, 3-amino-1,2-propanediol, 3-dimethylamino-1,2-propanediol and tris(hydroxymethylamino)methane.

Amino acids that may be used are of natural or synthetic origin, in their L, D or racemic form, and comprise at least one acid function chosen more particularly from carboxylic acid, sulfonic acid, phosphonic acid or phosphoric acid functions. The amino acids may be in neutral or ionic form.

As amino acids that may be used in the present invention, mention may be made especially of aspartic acid, glutamic acid, alanine, arginine, ornithine, citrulline, asparagine, carnitine, cysteine, glutamine, glycine, histidine, lysine, isoleucine, leucine, methionine, N-phenylalanine, proline, serine, taurine, threonine, tryptophan, tyrosine and valine.

It may be preferable that the amino acids are basic amino acids comprising an additional amine function optionally included in a ring or in an ureido function.

Such basic amino acids may preferably be chosen from those corresponding to formula (IV) below:

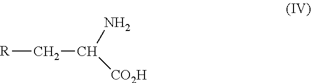

in which

R represents a group chosen from:

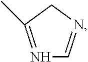

—(CH₂)₃—NH₂,

—(CH₂)₂—NH₂,

—(CH₂)₂—NH—CO—NH₂, and

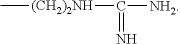

The compounds corresponding to formula (IV) include histidine, lysine, arginine, ornithine and citrulline.

The organic basifying agent may be chosen from organic amines of heterocyclic type. Besides histidine that has already been mentioned in the amino acids, mention may in particular be made of pyridine, piperidine, imidazole, triazole, tetrazole and benzimidazole.

The organic basifying agent may also be chosen from amino acid dipeptides. As amino acid dipeptides that may be used in the present invention, mention may be made especially of carnosine, anserine and baleine.

The organic basifying agent may also be chosen from compounds comprising a guanidine function. As amines of this type that may be used in the present invention, besides arginine, which has already been mentioned as an amino acid, mention may be made especially of creatine, creatinine, 1,1-dimethylguanidine, 1,1-diethyl-guanidine, glycocyamine, metformin, agmatine, N-amidinoalanine, 3-guanidino-propionic acid, 4-guanidinobutyric acid and 2-([amino(imino)methyl]amino)ethane-1-sulfonic acid.

In a preferred embodiment of the present invention, the organic basifying agent may be selected from amino acids, preferably basic amino acids, and more preferably arginine, lysine, histidine or mixtures thereof. Even more preferentially, the organic basifying agent may be arginine.

Hybrid compounds that may be mentioned include the salts of the amines mentioned previously with acids such as carbonic acid or hydrochloric acid. Guanidine carbonate or monoethanolamine hydrochloride may be used in particular.

The (2) pH adjusting agent may be present in an amount of 0.01% by weight or more, preferably 0.05% by weight or more, and more preferably 0.1% by weight or more, relative to the total weight of the composition.

The (2) pH adjusting agent may be present in an amount of 15% by weight or less, preferably 10% by weight or less, and more preferably 5% by weight or less, relative to the total weight of the composition.

The (2) pH adjusting agent may be present in an amount ranging from 0.01% to 15% by weight, preferably from 0.05% to 10% by weight, and more preferably from 0.1% to 5% by weight or less, relative to the total weight of the composition.

It is preferable that the composition according to the present invention has a pH of 4.5 or more, and more preferably 5 or more.

It is preferable that the composition according to the present invention has a pH of 6.5 or less, and more preferably 6 or less.

It is preferable that the composition according to the present invention has a pH of from 4.5 to 6.5, and more preferably from 5 to 6.

The pH of the composition means the pH of the aqueous phase of the composition according to the present invention. The pH can be measured in accordance with JIS Z 8802 (2011).

It may be preferable that at least one buffer or buffering agent also be used, as the (2) pH adjusting agent, in combination with the acidifying agent and/or the basifying agent, in order to stabilize the pH of the composition according to the present invention.

As the buffer, any of commonly known buffers may be used. For example, salts of acids or bases, preferably salts of weak acids or weak bases, may be used. For example, sodium citrate or sodium lactate may be used as the buffer, if citric acid or lactic acid is used as the acidifying agent.

### Water

The composition according to the present invention comprises (3) water.

The amount of the (3) water in the composition according to the present invention may be 20% by weight or more, preferably 30% by weight or more, and more preferably 40% by weight or more, relative to the total weight of the composition.

On the other hand, the amount of the (3) water in the composition according to the present invention may be 99% by weight or less, preferably 95% by weight or less, and more preferably 90% by weight or less, relative to the total weight of the composition.

The amount of (3) water in the composition according to the present invention may be from 20% to 99% by weight, preferably from 30% to 95% by weight, and more preferably from 40% to 90% by weight, relative to the total weight of the composition.

### Optional Additives

The composition according to the present invention may also comprise any optional additive(s) usually used in the field of cosmetics, chosen, for example, from solvents, antioxidants, chelating agents, cosmetic active agents other than ingredient (1), such as oils, preservatives, and mixtures thereof.

It is a matter of routine operations for a person skilled in the art to adjust the nature and amount of the above optional additives which may be present in the composition in accordance with the present invention such that the desired cosmetic properties are not thereby affected.

As the solvents, mention may be made of one or several cosmetically acceptable organic solvents, which may be alcohols: in particular monovalent alcohols such as ethyl alcohol, isopropyl alcohol, benzyl alcohol, and phenylethyl alcohol; diols such as ethylene glycol, propylene glycol, and butylene glycol; other polyols such as glycerol, sugar, and sugar alcohols; and ethers such as ethylene glycol monomethyl, monoethyl, and monobutyl ethers, propylene glycol monomethyl, monoethyl, and monobutyl ether, and butylene glycol monomethyl, monoethyl, and monobutyl ethers.

The organic solvent(s) may be present in a concentration of from 0.01% to 30% by weight, preferably from 0.1% to 20% by weight, and more preferably from 1% to 10% by weight, relative to the total weight of the composition.

### Preparation

The composition according to the present invention can be prepared by mixing the above-described essential and optional ingredients in a conventional manner.

For example, the composition according to the present invention can be prepared by a process comprising the steps of mixing

(1) at least one thiopyridinone compound;

(2) at least one pH adjusting agent; and

(3) water.

It is possible to further mix any of the optional ingredients.

The mixing can be performed at any temperature such as room temperature (e.g., 20-25° C., preferably at 25° C.), preferably at a temperature of 30° C. or more, preferably 40° C. or more, and more preferably 50° C. or more. It is preferable to further mix with any of the above-described optional ingredients such as a pH adjusting agent.

The form of the composition according to the present invention is not particularly limited, and may take various forms such as a W/O emulsion, an O/W emulsion, a gel, a solution, or the like. It is preferable that the composition according to the present invention be in the form of an emulsion, preferably an O/W emulsion, and more preferably an O/W gel emulsion.

### Cosmetic Process

The composition according to the present invention may be used as a cosmetic or dermatologic composition, preferably a cosmetic composition, and more preferably a cosmetic composition for a keratin substance. As the keratin substance, mention may be made of the skin, scalp, hair, mucosa such as lips, and nails.

The composition according to the present invention may be used as a depigmenting, bleaching or whitening product for a keratinous substance such as skin. In particular, the composition according to the present invention may be used as a whitening product.

The composition according to the present invention may preferably be intended for application onto a keratin substance such as the skin, scalp and/or the lips, preferably the skin.

Thus, the composition according to the present invention can be used for a cosmetic process for a keratin substance, preferably the skin. In one embodiment, the present invention relates to a cosmetic process, preferably a whitening process, for a keratin substance, preferably skin, comprising the step of applying onto the keratin substance the composition according to the present invention.

The composition according to the present invention can be used as a topical cosmetic composition in the form of a lotion, a milky lotion, a cream, a gel, a paste, a serum, foam, or spray.

### Use

The present invention also relates to a use of (2) at least one pH adjusting agent in a composition comprising (1) at least one compound selected from compounds of formula (I) below, tautomers of formula (I′) below, salts thereof, solvates, such as hydrates, thereof, optical isomers thereof, racemates thereof, and mixtures thereof:

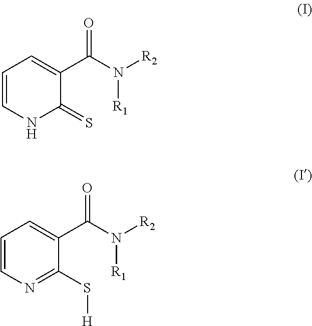

in which

R₁ denotes a radical chosen from
a) a hydrogen atom, and
b) a saturated, linear C₁-C₁₀ or branched C₃-C₁₀ alkyl group optionally substituted with one or more groups, which may be identical or different, chosen from
   i) —O—R₃, and
   ii) —S—R₃,

and

R₂ denotes a radical chosen from
a) a hydrogen atom,
b) a saturated, linear C₁-C₁₂ or branched C₃-C₁₂ or cyclic C₃-C₈ hydrocarbonated group optionally substituted with one or more groups, which may be identical or different, chosen from
   i) —O—R₃,
   ii) —S—R₃,
   iii) —C(O)—O—R₃, and
   iv) a C₅-C₁₂ aryl group optionally substituted with one or more hydroxyls and/or with one or more C₁-C₈ alkoxy radicals, and
c) a C₅-C₁₂ aryl group optionally substituted with one or more hydroxyls and/or with one or more C₁-C₈ alkoxy radicals

wherein

R₃ denotes a radical chosen from
a) a hydrogen atom, and
b) a saturated, linear C₁-C₁₀ or branched C₃-C₁₀ alkyl group in order to control the pH of the composition to be from 4.5 to 6.5 for stabilizing the (1) compound(s).

The term “stabilize” has the same meaning as enhancing stability.

The use according to the present invention can increase the stability of the (1) thiopyridinone compound in the composition comprising the same.

Therefore, the use according to the present invention can make it possible to store a composition comprising the (1) thiopyridinone compound for a long period of time, and in particular even under hot conditions.

The above explanations regarding the (1) thiopyridinone compound and the (2) pH adjusting agent for the compositions according to the present invention can also apply to those used in the use according to the present invention.

The composition in the use according to the present invention may include any of the optional ingredients as explained above for the compositions according to the present invention.

### EXAMPLES

The present invention will be described in a more detailed manner by way of examples. However, these examples should not be construed as limiting the scope of the present invention.

### Examples 1-3 and Comparative Example 1

### Preparation

Each of the compositions according to Examples 1-3 and Comparative Example 1 was prepared by mixing the ingredients shown in Table 1. The numerical values for the amounts of the ingredients are all based on “% by weight” as active materials.

TABLE 1

| | | | | Comp. |
|---|---|---|---|---|
| Ingredients | Ex. 1 | Ex. 2 | Ex. 3 | Ex. 1 |
| Water | qsp 100 | qsp 100 | qsp 100 | qsp 100 |
| Glycerin | 5.2 | 5.2 | 5.2 | 5.2 |
| Butylene Glycol | 4.4 | 4.4 | 4.4 | 4.4 |
| Preservatives | 1.3 | 1.3 | 1.3 | 1.3 |
| N-[(2-thioxo-1,2- | 1.0 | 1.0 | 1.0 | 1.0 |
| dihydropyridin-3- | | | | |
| yl)carbonyl]glycine | | | | |
| Potassium Hydroxide | 0.52 | — | — | 0.54 |
| Sodium Hydroxide | — | 0.70 | — | — |
| Arginine | — | — | 0.70 | — |
| pH | 5.8 | 5.8 | 5.8 | 7.0 |
| Thiopyridinone | 95 | 95 | 95 | 41 |
| Compound | | | | |
| Remaining Rate (%) | | | | |
| Stability | Very | Very | Very | Poor |
| | Good | Good | Good | |

N-[(2-thioxo-1,2-dihydropyridin-3-yl)carbonyl]glycine (2-MERCAPTONICOTINOYL GLYCINE): Compound 20

### Evaluations

pH

The pH at 25° C. of each of the compositions according to Examples 1-3 and Comparative Example 1 was measured as follows.

### Equipment

1. pH meter; Laqua F-71 (Horiba)

2. pH electrodes; 9615S-10D (Horiba)

3. pH standard solutions (Horiba)

4. pH probe internal solution (Horiba)

### Procedures

1. The internal solution filler ports of the pH meter were opened, and internal solutions were provided around the electrodes.

2. Calibration was performed using standard solutions at pH 9, 7 and 4.

3. The electrodes were washed with deionized water.

4. The protective cap of one of the electrodes (glass electrode) was removed, and the electrodes were immersed into each of the compositions according to Examples 1-3 and Comparative Example 1.

5. The pH was recorded after the pH value was stabilized.

The results are shown in the “pH” line in Table 1.

### Thiopyridinone Compound Remaining Rate

The amount of the thiopyridinone compound in each of the compositions according to Examples 1-3 and Comparative Example 1 was measured by an HPLC-UV assay at the following timing.

Timing (1) Just after the preparation of the composition (TO)

Timing (2) 2 weeks after the preparation, where the composition was maintained in a sealed and light-shielding container at 55° C.

The details of the HPLC-UV assay are as follows.

### Apparatus/Reagents

| | |
|---|---|
| HPLC System | Ultra performance LC (UPLC) |
| HPLC Column | RP18, 1.7 μm, 2.1 mm × 100 mm |
| Eluent A | 0.1% Phosphoric acid in water |
| Eluent B | Methanol |

### HPLC Conditions

| | |
|---|---|
| UV detector | 296 nm |
| Column Temp. | 30° C. |
| Flow Rate | 0.4 mL/min |
| Injection Volume | 1 μL |
| Mobile Phase | Gradient mode |
| | A: 0.1% Phosphoric acid in water |
| | B: Methanol |

The thiopyridinone compound remaining rate was determined by the following equation:

Thiopyridinone Compound Remaining Rate (%)=Amount of Thiopyridinone Compound at Timing (2)/Amount of Thiopyridinone Compound at Timing (1)

The results are shown in the “Thiopyridinone Compound Remaining Rate (%)” line in Table 1.

The thiopyridinone compound remaining rate was also categorized in accordance with the following criteria:

Very Good: 90% or more

Good: 50% or more and less than 90%

Poor: less than 50%

The results are shown in the “Stability” line in Table 1.

### Results

The compositions according to Examples 1-3 each of which included a thiopyridinone compound and a pH adjusting agent to control the pH of the composition from 4.5 to 6.5 were more stable, even under elevated temperature, such that most of the thiopyridinone compound remained, than the composition according to Comparative Example 1 which had a pH of more than 6.5.

1. A composition comprising
(1) at least one compound selected from compounds of formula (I) below, tautomers of formula (I′) below, salts thereof, solvates thereof, optical isomers thereof, racemates thereof, and mixtures thereof: 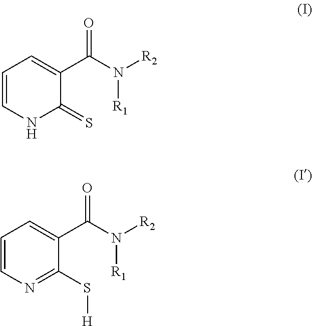
in which
R₁ denotes a radical chosen from
a) a hydrogen atom, and
b) a saturated, linear C₁-C₁₀ or branched C₃-C₁₀ alkyl group optionally substituted with one or more groups, which may be identical or different, chosen from
i) —O—R₃, and
ii) —S—R₃,
and
R₂ denotes a radical chosen from
a) a hydrogen atom,
b) a saturated, linear C₁-C₁₂ or branched C₃-C₁₂ or cyclic C₃-C₈ hydrocarbonated group optionally substituted with one or more groups, which may be identical or different, chosen from
i) —O—R₃,
ii) —S—R₃,
iii) —C(O)—O—R₃, and
iv) a C₅-C₁₂ aryl group optionally substituted with one or more hydroxyls and/or with one or more C₁-C₈ alkoxy radicals, and
c) a C₅-C₁₂ aryl group optionally substituted with one or more hydroxyls and/or with one or more C₁-C₈ alkoxy radicals
wherein
R₃ denotes a radical chosen from
a) a hydrogen atom, and
b) a saturated, linear C₁-C₁₀ or branched C₃-C₁₀ alkyl group;
and
(2) at least one pH adjusting agent, and
(3) water,
wherein
the pH of the composition is from 4.5 to 6.5.

2. The composition according to claim 1, wherein:
R₁ of formula (I) and (I′) represents a hydrogen atom;
or
R₁ of formula (I) and (I′) represents a linear (C₁-C₁₀) alkyl group or a branched (C₃-C₁₀) alkyl group.

3. The composition according to claim 1, wherein:
R₂ of formula (I) and (I′) represents a hydrogen atom;
or
R₂ of formula (I) and (I′) represents a linear (C₁-C₁₀) alkyl group or a branched (C₃-C₁₀) alkyl group; the said alkyl group of R₂ being not substituted.

4. The composition according to claim 1, wherein:
R₂ of formula (I) and (I′) represents a linear (C₁-C₁₀) alkyl group or a branched (C₃-C₁₀) alkyl group; the said alkyl group being substituted by one or more groups selected from i), ii), iii) and iv) as defined above.

5. The composition according to claim 1, wherein:
R₂ of formula (I) and (I′) represents a (C₃-C₈) cycloalkyl group;
or
R₂ of formula (I) and (I′) represents a C₅-C₁₂ aryl group optionally substituted with one or more hydroxyls and/or with one or more C₁-C₈ alkoxy radicals.

6. The composition according to claim 1, wherein:
R₃ of formula (I) and (I′) represents a hydrogen atom;
or
R₃ of formula (I) and (I′) represents a saturated, linear C₁-C₁₀ or branched C₃-C₁₀ alkyl group.

7. The composition according to claim 1, wherein:
R₁ of formula (I) and (I′) represents a radical chosen from
a) a hydrogen atom, and
b) a saturated, linear C₁-C₆ or branched C₃-C₆ alkyl group optionally substituted with one or more groups, which may be identical or different, chosen from
i) —O—R₃, and
ii) —S—R₃;
R₂ of formula (I) and (I′) represents a radical chosen from
a) a hydrogen atom, and
b) a saturated, linear C₁-C₁₀ or branched C₃-C₁₀ or cyclic C₃-C₈ hydrocarbonated group, optionally substituted with one or more groups, which may be identical or different, chosen from
i) —O—R₃,
ii) —S—R₃,
iii) —C(O)—O—R₃, and
iv) a phenyl group optionally substituted with one or more hydroxyls and/or with one or more C₁-C₄ alkoxy radicals; and
R₃ of formula (I) and (I′) represents a radical chosen from
a) a hydrogen atom, and
b) a saturated, linear C₁-C₆ or branched C₃-C₆ alkyl group,

8. The composition according to claim 1, wherein:
the (1) compound is selected from the compounds 1 to 24 below, tautomers thereof, salts thereof, solvates thereof, optical isomers thereof, racemates thereof, and mixtures thereof:

| No. | Structure | ChemicalName |
|---|---|---|
| 1 | 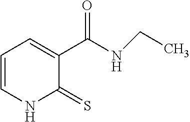 | N-ethyl-2-thioxo-1,2- dihydropyridine-3- carboxamide |
| 2 | 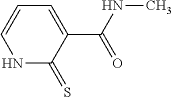 | N-methyl-2-thioxo-1,2- dihydropyridine-3- carboxamide |
| 3 | 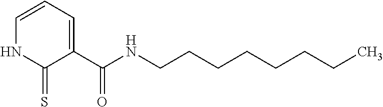 [SMILES: CCCCCCCCNC(=O)c1ccc[nH]c1=S] | N-octyl-2-thioxo-1,2- dihydropyridine-3- carboxamide |
| 4 | 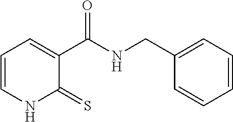 [SMILES: O=C(NCc1ccccc1)c1ccc[nH]c1=S] | N-benzyl-2-thioxo-1,2- dihydropyridine-3- carboxamide |
| 5 | 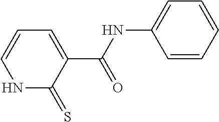 [SMILES: O=C(Nc1ccccc1)c1ccc[nH]c1=S] | N-phenyl-2-thioxo-1,2- dihydropyridine-3- carboxamide |
| 6 | 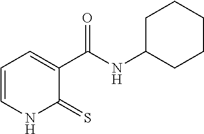 [SMILES: O=C(NC1CCCCC1)c1ccc[nH]c1=S] | N-cyclohexyl-2-thioxo- 1,2-dihydropyridine-3- carboxamide |
| 7 | 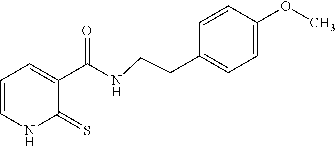 [SMILES: COc1ccc(CCNC(=O)c2ccc[nH]c2=S)cc1] | N-[2-(4- methoxyphenyl)ethyl]- 2-thioxo-1,2- dihydropyridine-3- carboxamide |
| 8 | 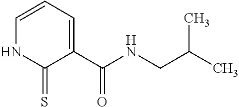 | N-(2-methylpropyl)-2- thioxo-1,2- dihydropyridine-3- carboxamide |
| 9 | 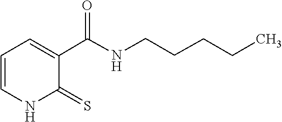 | N-pentyl-2-thioxo-1,2- dihydropyridine-3- carboxamide |
| 10 | 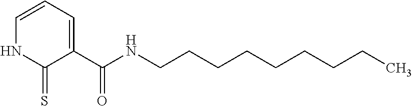 [SMILES: CCCCCCCCCNC(=O)c1ccc[nH]c1=S] | N-nonyl-2-thioxo-1,2- dihydropyridine-3- carboxamide |
| 11 | 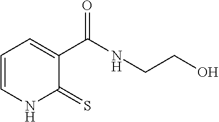 [SMILES: O=C(NCCO)c1ccc[nH]c1=S] | N-(2-hydroxyethyl)-2- thioxo-1,2- dihydropyridine-3- carboxamide |
| 12 | 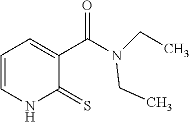 | N,N-diethyl 2- mercaptonicotinamide |
| 13 | 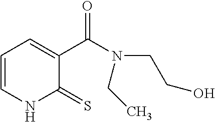 | N-ethyl-N-(2- hydroxyethyl)-2- thioxo-1,2- dihydropyridine-3- carboxamide |
| 14 | 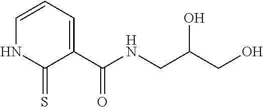 [SMILES: O=C(NCC(O)CO)c1ccc[nH]c1=S] | N-(2,3- dihydroxypropyl)-2- thioxo-1,2- dihydropyridine-3- carboxamide |
| 15 | 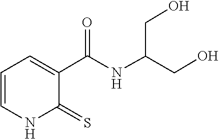 [SMILES: O=C(NC(CO)CO)c1ccc[nH]c1=S] | N-(1,3- dihydroxypropan-2-yl)- 2-thioxo-1,2- dihydropyridine-3- carboxamide |
| 16 | 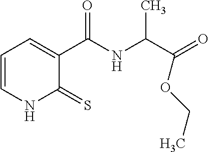 [SMILES: CCOC(=O)C(C)NC(=O)c1ccc[nH]c1=S] | Ethyl N-[(2-thioxo- 1,2-dihydropyridin-3- yl)carbonyl]alaninate |
| 17 | 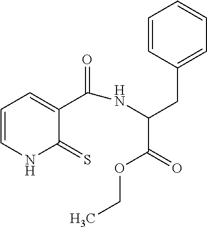 [SMILES: CCOC(=O)C(Cc1ccccc1)NC(=O)c1ccc[nH]c1=S] | Ethyl N-[(2-thioxo- 1,2-dihydropyridin-3- yl)carbonyl]phenyl alaninate |
| 18 | 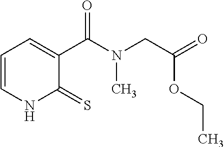 [SMILES: CCOC(=O)CN(C)C(=O)c1ccc[nH]c1=S] | Ethyl N-methyl-N-[(2- thioxo-1,2- dihydropyridin-3- yl)carbonyl]glycinate |
| 19 | 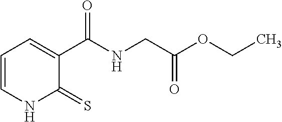 [SMILES: CCOC(=O)CNC(=O)c1ccc[nH]c1=S] | Ethyl N-[(2-thioxo- 1,2-dihydropyridin-3- yl)carbonyl]glycinate |
| 20 | 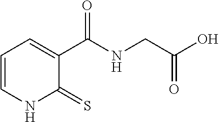 [SMILES: O=C(O)CNC(=O)c1ccc[nH]c1=S] | N-[(2-thioxo-1,2- dihydropyridin-3- yl)carbonyl]glycine |
| 21 | 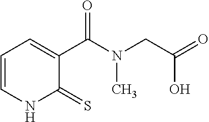 | N-methyl-N-[(2-thioxo- 1,2-dihydropyridin-3- yl)carbonyl]glycine |
| 22 | 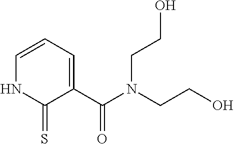 [SMILES: O=C(c1ccc[nH]c1=S)N(CCO)CCO] | N,N-bis(2- hydroxyethyl)-2- thioxo-1,2- dihydropyridine-3- carboxamide |
| 23 | 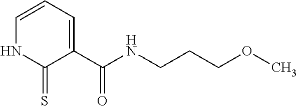 | N-(3-methoxypropyl)-2- thioxo-1,2- dihydropyridine-3- carboxamide |
| 24 | 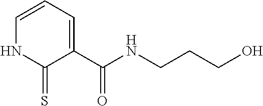 [SMILES: O=C(NCCCO)c1ccc[nH]c1=S] | N-butyl-2-thioxo-1,2- dihydropyridine-3- carboxamide |

9. The composition according to claim 1, wherein the amount of the (1) compound(s) in the composition is from 0.01% to 10% by weight relative to the total weight of the composition.

10. The composition according to claim 1, wherein the (2) pH adjusting agent is selected from acidifying agents, basifying agents, and mixtures thereof.

11. The composition according to claim 1, wherein the amount of the (2) pH adjusting agent(s) in the composition is from 0.01% to 15% by weight relative to the total weight of the composition.

12. The composition according to claim 1, wherein the amount of the (3) water in the composition is from 20% to 99% by weight relative to the total weight of the composition.

13. The composition according to claim 1, wherein the composition is for whitening a keratin substance.

14. A cosmetic process for a keratin substance, comprising:
applying to the keratin substance the composition according to claim 1.

15. A method for stabilizing at least one compound selected from compounds of formula (I), comprising:
combining (2) at least one pH adjusting agent with a composition comprising (1) at least one compound selected from compounds of formula (I) below, tautomers of formula (I′) below, salts thereof, solvates thereof, optical isomers thereof, racemates thereof, and mixtures thereof: 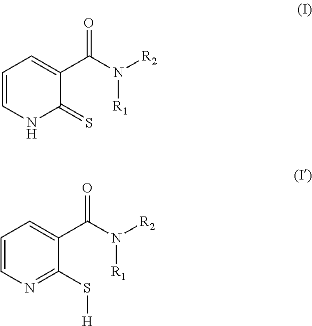
in which
R₁ denotes a radical chosen from
a) a hydrogen atom, and
b) a saturated, linear C₁-C₁₀ or branched C₃-C₁₀ alkyl group optionally substituted with one or more groups, which may be identical or different, chosen from
i) —O—R₃, and
ii) —S—R₃,
and
R₂ denotes a radical chosen from
a) a hydrogen atom,
b) a saturated, linear C₁-C₁₂ or branched C₃-C₁₂ or cyclic C₃-C₈ hydrocarbonated group optionally substituted with one or more groups, which may be identical or different, chosen from
i) —O—R₃,
ii) —S—R₃,
iii) —C(O)—O—R₃, and
iv) a C₅-C₁₂ aryl group optionally substituted with one or more hydroxyls and/or with one or more C₁-C₈ alkoxy radicals, and
c) a C₅-C₁₂ aryl group optionally substituted with one or more hydroxyls and/or with one or more C₁-C₈ alkoxy radicals
wherein
R₃ denotes a radical chosen from
a) a hydrogen atom, and
b) a saturated, linear C₁-C₁₀ or branched C₃-C₁₀ alkyl group in order to control the pH of the composition to be from 4.5 to 6.5.
